(19) United States
(12) Patent Application Publication
(10) Pub. No.: US 2015/0144558 A1
(43) Pub. Date: May 28, 2015

(54) SLOW DIALYSATE ADAPTOR APPARATUS FOR INTERMITTENT HEMODIALYSIS

(71) Applicant: THE REGENTS OF THE UNIVERSITY OF CALIFORNIA, Oakland, CA (US)

(72) Inventor: Larry D. Cowgill, Sacramento, CA (US)

(73) Assignee: THE REGENTS OF THE UNIVERSITY OF CALIFORNIA, Oakland, CA (US)

(21) Appl. No.: 14/549,810

(22) Filed: Nov. 21, 2014

Related U.S. Application Data
(63) Continuation of application No. PCT/US2013/044609, filed on Jun. 6, 2013.
(60) Provisional application No. 61/656,296, filed on Jun. 6, 2012.

(51) Int. Cl. A61M 1/16

(52) U.S. Cl. CPC A61M 1/1603; A61M 2205/3334
USPC 210/646; 210/137; 210/416.1

(57) ABSTRACT

A system and method for providing slow dialysate flow with an intermittent hemodialysis delivery (IHD) system are disclosed. The system includes a dialysate slow-flow fluid pump having an outlet for connection to an inlet port of a hemodialyzer and an inlet for connection to a dialysate bulk flow line in association with the IHD. The system further includes an outlet line for coupling an outlet port of the hemodialyzer back to the dialysate bulk flow line, wherein the dialysate slow-flow fluid pump is configured to control the rate of delivery of dialysate to the hemodialyzer.

### CROSS-REFERENCE TO RELATED APPLICATIONS

This application is a 35 U.S.C. §111(a) continuation of PCT international application number PCT/US2013/044609 filed on Jun. 6, 2013, incorporated herein by reference in its entirety, which claims priority to, and the benefit of, U.S. provisional patent application Ser. No. 61/656,296 filed on Jun. 6, 2012, incorporated herein by reference in its entirety. Priority is claimed to each of the foregoing applications.

The above-referenced PCT international application was published as PCT International Publication No. WO 2013/184973 on Dec. 12, 2013, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED IN A COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND

1. Technical Field

This technology pertains generally to hemodialysis, and more particularly to a slow dialysate adaptor for intermittent hemodialysis.

2. Background Discussion

Conventional intermittent hemodialysis (IHD) delivery systems designed for adult human hemodialysis have a long established use and suitability for routine dialysis delivery to animal patients. However, they are not sufficiently flexible in operational controls to permit safe, precisely regulated, and extended slow rates of dialysis delivery to small and/or severely uremic animal (or human) patients. These therapeutic objectives are more commonly provided in human patients by continuous renal replacement therapies (CRRT) provided by separate delivery systems of inherently different design characteristics, which are not as universally suitable for animal hemodialysis.

For IHD, the rate of dialysis delivery is controlled by the rate at which blood is delivered to the hemodialyzer (the artificial kidney or blood filter). The requisite slow blood flow rate (1 to 10 ml/minute) required for slow dialysis delivery to small animal patients (<10 kg) is typically below the approved operational design of IHD delivery systems, and, if obtainable, such slow rates of blood flow predispose to clotting in the extracorporeal circuit during extended dialysis sessions.

### BRIEF SUMMARY

An aspect of the technology described herein is a device for controlling the rate of dialysis delivery by reducing the rate of dialysate flow independently from the blood flow rate, particularly in situations where the dialysate flow rate is considerably less than the blood flow rate and the dialysate in the hemodialyzer is at or near equilibrium with the blood compartment.

In one aspect, the technology described herein provides a diversionary dialysate circuit (or flow path) for bulk, on-line generated dialysate destined for delivery to a hemodialyzer in a conventional IHD configuration, yet allows for a controlled, slow-flow loop from the bulk dialysate that is delivered alternatively to the hemodialyzer. With use of the technology described herein, the rate of dialysis delivery can be slowed, while maintaining intact all the functionality of the IHD delivery system, including, but not limited to: (a) on-line dialysate generation from non sterile concentrates; (b) monitoring, regulation, and alarming of all systems; (c) control of ultrafiltration; (d) control of anticoagulant delivery; (e) blood and dialysate flow rates within the operational specifications of the delivery system; and (f) use of high blood flow rates to prevent clotting in the extracorporeal circuit.

In a preferred embodiment, the system of the technology described herein allows control of the rate of dialysis delivery (urea clearance) to any slow- (e.g. 1 to 20 ml/min) or intermediate- (e.g. 20-100 ml/min) intensity by the precise rate of dialysate flow provided through the controlled-flow loop.

The system and methods of the technology described herein include, but are not limited to the following features and benefits: (a) control and provide slow-flow dialysate delivery at fast blood flow rates, (b) provide protection from dialysis disequilibrium in small patients compared to conventional IHD procedures; (c) provide ability to use inexpensive, on-line generated, standard or ultrapure dialysate compared to expensive, pre-packaged, sterile dialysate solutions required for CRRT; (d) provide for a single IHD platform (machine) to provide both conventional IHD (for usual applications) and prolonged slow low efficiency dialysis (SLED) treatments for equivalent CRRT prescriptions in small and severely uremic patients; (e) minimize anticoagulant requirement due to ability to maintain fast blood flow rates; (f) provide accurate prediction of dialyzer clearance and hence dialysis prescription and delivered dialysis dose since, at slow dialysate flow, the flow rate is equivalent to the clearance of the hemodialyzer; (g) if equipped with inlet and outlet flow monitors, provide precise rates of net ultrafiltration that are measurable in real-time; (h) require no internal or external modification of the standard IHD delivery system; and (i) permit a smaller volume extracorporeal blood path than current CRRT platforms

Further aspects of the technology will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a schematic diagram showing an embodiment of the inventive adapter of the technology described herein connected between the dialysate bulk flow and hemodialyzer.

FIG. 2 is a schematic diagram of an alternative embodiment of the adapter shown in FIG. 1.

FIG. 3 is a flow diagram of a method of controlling the rate of dialysis delivery in accordance with the technology described herein.

FIG. 4 is a set of graphs showing changes in BUN before and during 300 minutes of hemodialysis using the system of the technology described herein at a slow-flow dialysate rate of 3 ml/min in a 2.9 kg cat.

FIG. 5 is a graph showing changes in BUN before and during 420 minutes of hemodialysis using the system of the technology described herein at a slow-flow dialysate rate of 3 ml/min in a 2.8 kg cat.

FIG. 6A and FIG. 6B are plots showing urea, nitrogen and creatinine concentration, respectively, before and during 400 minutes of hemodialysis using the system of the technology described herein at a slow dialysate flow rate of 4 mL/min and blood flow rate of 32 mL/min in a 5 kg cat.

FIG. 7 is a graph showing the ex vitro dialyzer clearance (K_{d}) of FD&C Blue1 (792 Da, squares) and the in vivo dialyzer clearance of urea nitrogen (triangles) and creatinine (circles) in animal-patients dialyzed with the technology described herein.

FIG. 8 is a plot showing the real-time changes in FD&C Blue 1 during ex vivo hemodialysis experiments in simulated patients.

### DETAILED DESCRIPTION

The technology described herein is directed to a dialysate diversion circuit 10 having a regulated slow-flow loop to control delivery of dialysate to a conventional hemodialyzer (HD) 14 (e.g. an intermittent hemodialyzer or similar device using online-generated bulk dialysate that may comprise standard or ultrapure dialysate) for prolonged slow low efficiency dialysis (SLED). It is appreciated that the technology described herein may comprise a dialysate diversion circuit 10 that is a standalone device for attachment to an existing hemodialyzer 14 and dialysate bulk flow 12, or may comprise a hemodialysis system 50 configured for to provide both conventional IHD (for usual applications) and prolonged slow, low-efficiency dialysis (SLED) treatments for equivalent CRRT prescriptions in small and severely uremic patients.

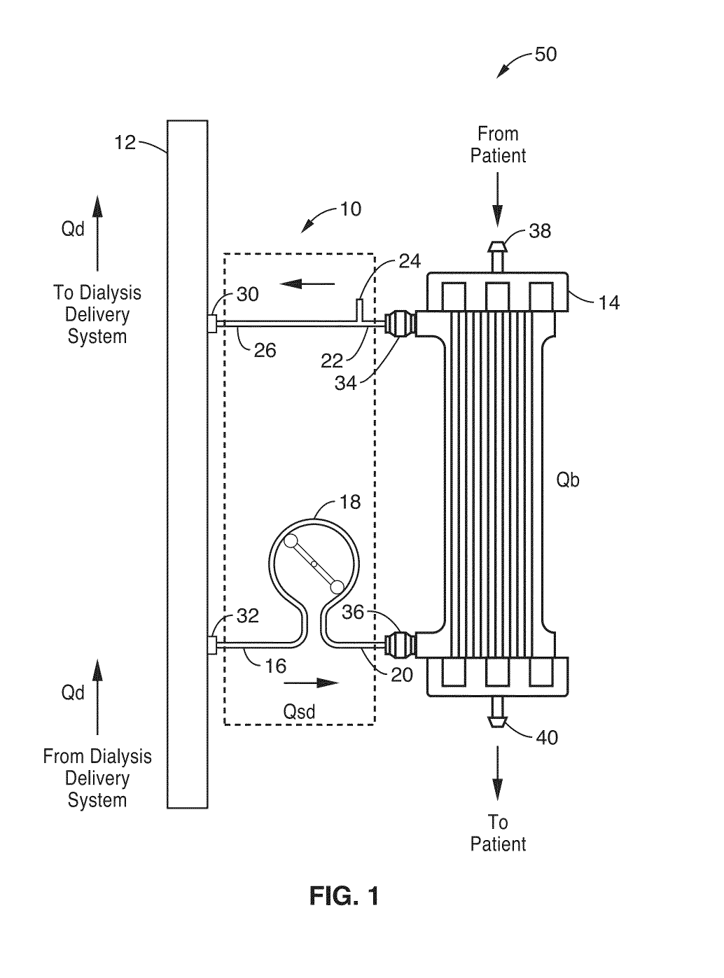

In the embodiment shown in FIG. 1, the dialysate diversion circuit 10 is shown connected to the online-generated dialysate bulk flow 12 (flowing at rate Q_{d}) and the hemodialyzer (HD) 14. The components of the dialysate diversion circuit 10 comprise (in the direction of flow) a male inlet connector 16 configured to couple to the female dialysate connector 32 from the IHD delivery system dialysate bulk flow 12, which directs dialysate at flow rate Qd to an input of dialysate slow-flow fluid pump 18. The output of the pump, which is at a controlled slow-flow rate Q_{sd}, is then directed via female connector 20 for attachment to the conventional male inlet port 36 of the HD 14 at the blood outlet end 40 of the hemodialyzer 14 (with blood flowing at rate Q_{b}). In a preferred embodiment, the fluid pump 18 is a variable-speed pump capable at operating at slow-flow rates Q_{sd} below 100 ml/min, preferably below 20 ml/min, and more preferably below 10 ml/min.

Dialysate diversion circuit 10 further includes an outlet line having a female outlet dialysate connector 22 for attachment to the conventional male outlet port 34 on the hemodialyzer 14 at the blood inlet end 38 of the hemodialyzer 14, and male outlet dialysate connector 26 for attachment to the conventional female dialysate connector 30 from the IHD delivery system.

Dialysate diversion circuit 10 may further include a “T” dialysate sampling port 24, between connector 22 and outlet connection 26 to the dialysate bulk flow. Sampling port 24 may be used to for temperature sensing, sample collection, etc.

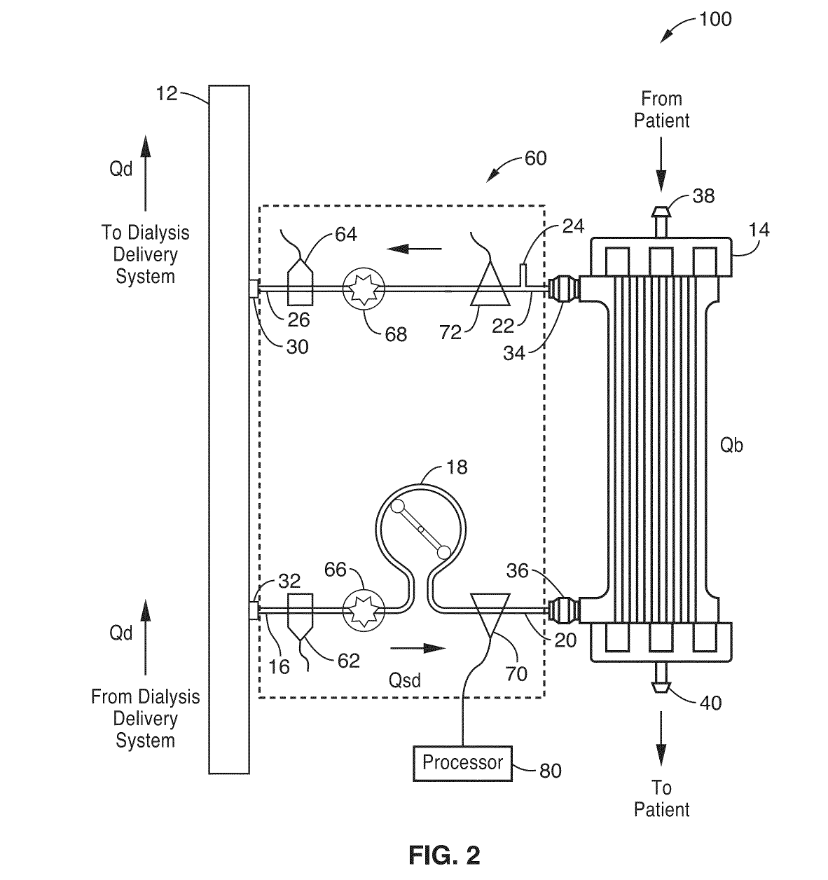

FIG. 2 is a schematic diagram of an alternative dialysate diversion circuit 60 having a regulated slow-flow loop to control delivery of dialysate to a conventional hemodialyzer (HD) 14 (e.g. an intermittent hemodialyzer using online-generated bulk dialysate that may comprise standard or ultrapure dialysate) for prolonged slow low efficiency dialysis (SLED). It is appreciated that the technology described herein may comprise a dialysate diversion circuit 60 that is a standalone device for attachment to an existing hemodialyzer 14 and dialysate bulk flow 12, or may comprise a hemodialysis system 100 configured for to provide both conventional IHD (for usual applications) and prolonged slow low efficiency dialysis (SLED) treatments for equivalent CRRT prescriptions in small and severely uremic patients.

The components of the dialysate diversion circuit 60 comprise (in the direction of flow) a male inlet connector 16 configured to couple to the female dialysate connector 32 from the IHD delivery system dialysate bulk flow 12, which directs bulk dialysate at flow rate Q_{d} to an input of dialysate slow-flow fluid pump 18. Between the inlet connector 16 and pump 18 are inlet conductivity monitor 62 and inlet flow sensor 66 (which may be mechanical or ultrasonic) to monitor the inlet dialysate conductivity and flow rate, respectively, in the slow-flow dialysate loop 60.

The output of the pump, which is at a controlled slow-flow rate Q_{sd}, is then directed via female connector 20 for attachment to the conventional male inlet port 36 of the HD 14 at the blood outlet end 40 of the hemodialyzer 14 (with blood flowing at rate Q_{b}). In a preferred embodiment, the fluid pump 18 is a variable-speed pump capable at operating at slow-flow rates Q_{sd} below 100 ml/min, preferably below 20 ml/min, and more preferably below 10 ml/min. Between the pump 18 and outlet connector 16 is an inlet dialysate pressure sensor 70 to measure inlet pressure in the slow-flow dialysate loop.

Dialysate diversion circuit 60 further includes a female outlet dialysate connector 22 for attachment to the conventional male outlet port 34 on the hemodialyzer 14 at the blood inlet end 38 of the hemodialyzer 14, sampling port 24, and male outlet dialysate connector 26 for attachment to the conventional female dialysate connector 30 from the IHD delivery system. Outlet pressure sensor 72, outlet flow sensor 68, and outlet conductivity sensor 64 may also be included between connectors 22 and 26 to monitor the outlet dialysate pressure, flow rate, and conductivity, respectively, in the slow-flow dialysate loop 60.

As shown in FIG. 2, one or more of the inlet conductivity monitor 62, inlet flow sensor 66 outlet pressure sensor 72, inlet dialysate pressure sensor 70, outlet flow sensor 68, and outlet conductivity sensor 64 may be coupled to a processor 80 for monitoring the flow in the circuit. Data from one or more of the sensors 62 through 72 may be used as feedback for modifying settings on the pump 18 (e.g. varying flow rate, etc.) according to a set flow-rate or pressure.

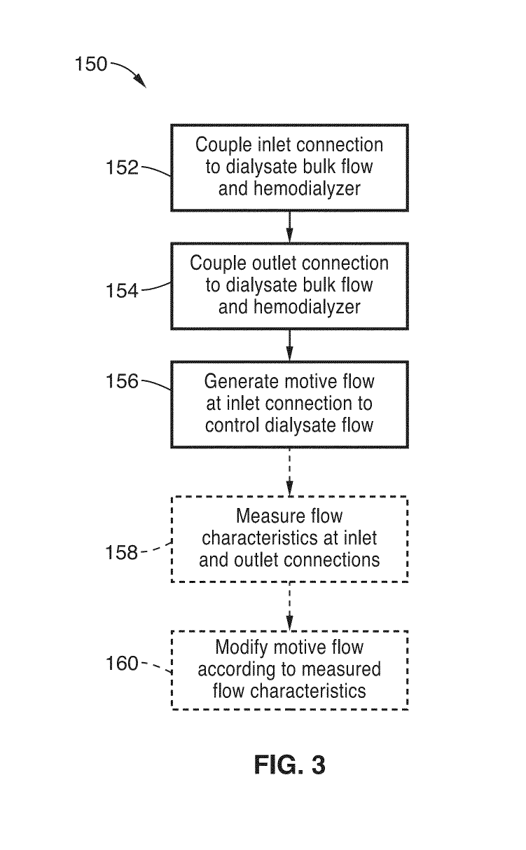

FIG. 3 shows a flow diagram of a method 150 of controlling the rate of dialysis delivery in accordance with the technology described herein. In block 152, an inlet connection (16, 20) comprising a pump 18 is coupled to the dialysate bulk flow 12 and HD 14. At block 154, an outlet connection (22, 26) is coupled to the dialysate bulk flow 12 and HD 14. Next at block 156, a motive flow (e.g. via pump 18) is generated at the input 20 to control dialysate flow into the HD 14.

Optionally, at block 158, one or more flow characteristics, such as pressure, conductivity and flow rate, may be measured at the inlet or outlet connections to obtain data with respect to the diverted dialysate flow. Furthermore, the motive flow may be modified at block 160 according to one or more of the measured flow characteristics.

Experiments were conducted on the system 50 shown in FIG. 1 to test the efficacy of the system and methods of the technology described herein, as follows:

### EXAMPLE 1

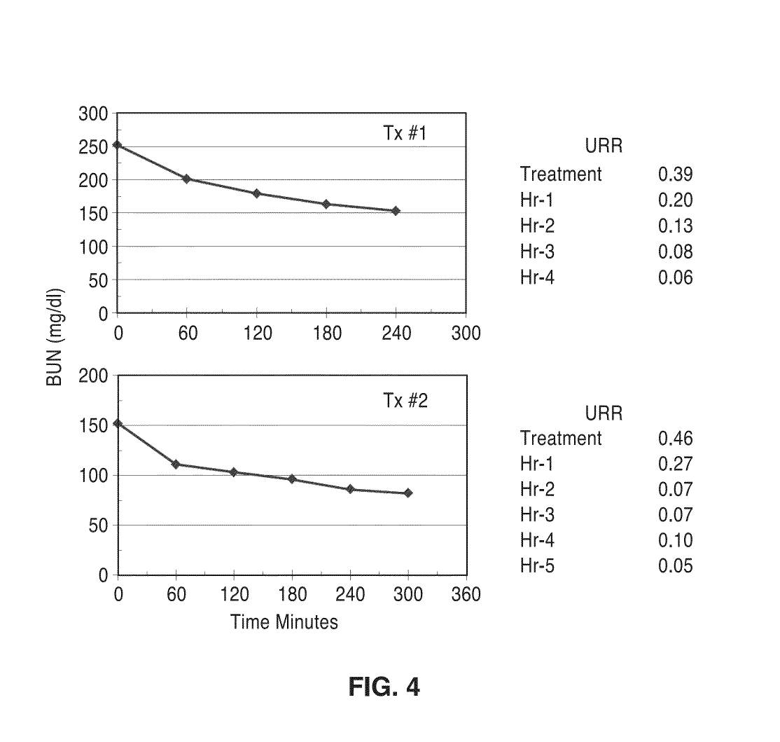

FIG. 4 shows a set of plots and Urea Reduction Ratio (URR) values for two treatments done on a first patient. The left portion of FIG. 3 shows changes in BUN before and during 300 minutes of hemodialysis for each test using the system 50 of the technology described herein at a slow-flow dialysate rate of 3 ml/min in a 2.9 kg cat. The right portion of FIG. 4 shows the Urea Reduction Ratio (URR) for each treatment hour and for the cumulative treatment.

### EXAMPLE 2

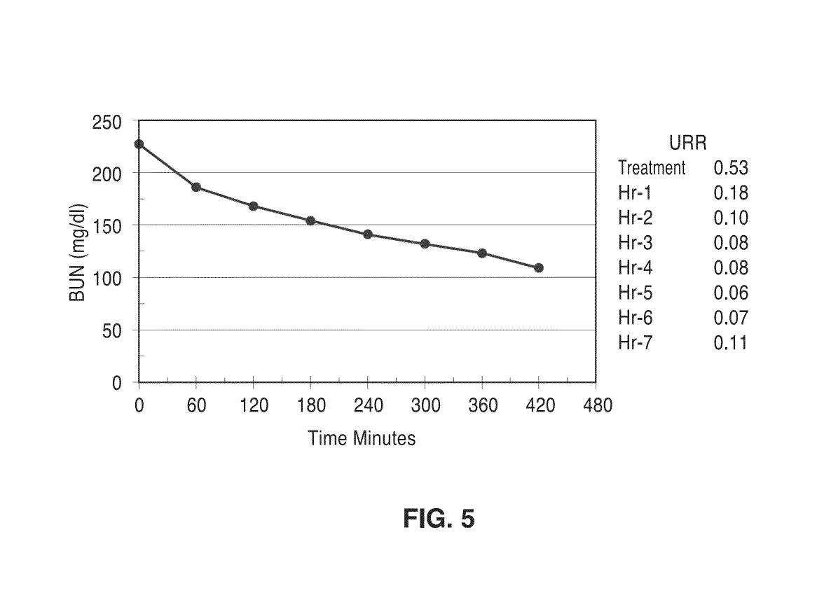

FIG. 5 shows a set of plots and URR values for one treatment done on a second patient. The left portion of FIG. 5 shows changes in BUN before and during 420 minutes of hemodialysis using the technology described herein at a slow-flow dialysate rate of 3 ml/min in a 2.8 kg cat. The right portion of FIG. 5 shows the Urea Reduction Ratio (URR) for each treatment hour and for the cumulative treatment.

### EXAMPLE 3

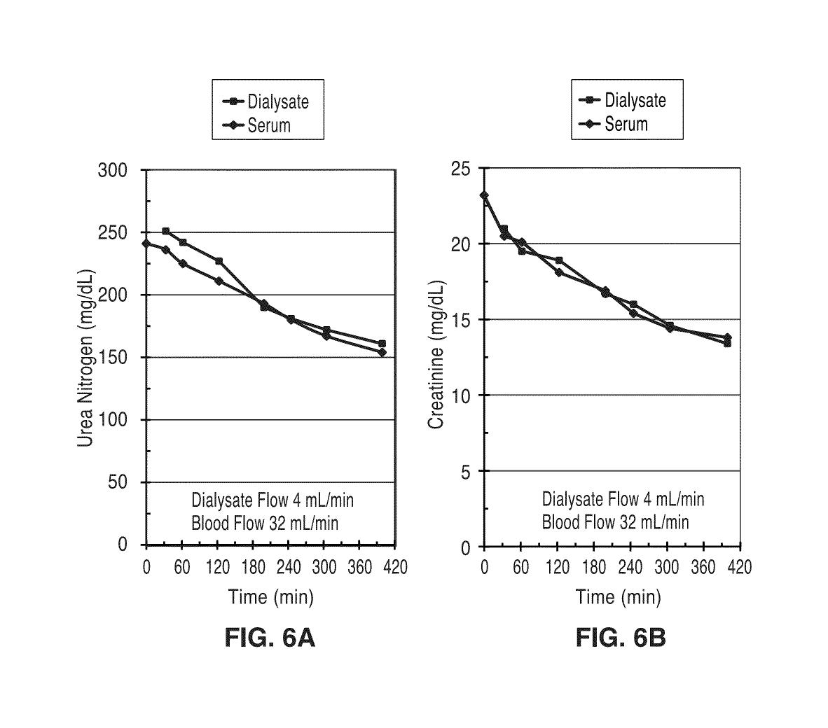

FIG. 6A and FIG. 6B are plots illustrating changes in serum and exit dialysate urea nitrogen (FIG. 6A) and creatinine (FIG. 6B) concentration before and during 400 minutes of hemodialysis using the system of the technology described herein at a slow dialysate flow rate of 4 mL/min and blood flow rate of 32 mL/min in a 5 kg cat. The inlet serum and exit dialysate solute concentrations were equivalent at each time point, indicating the solute equilibrium with that in the blood compartment.

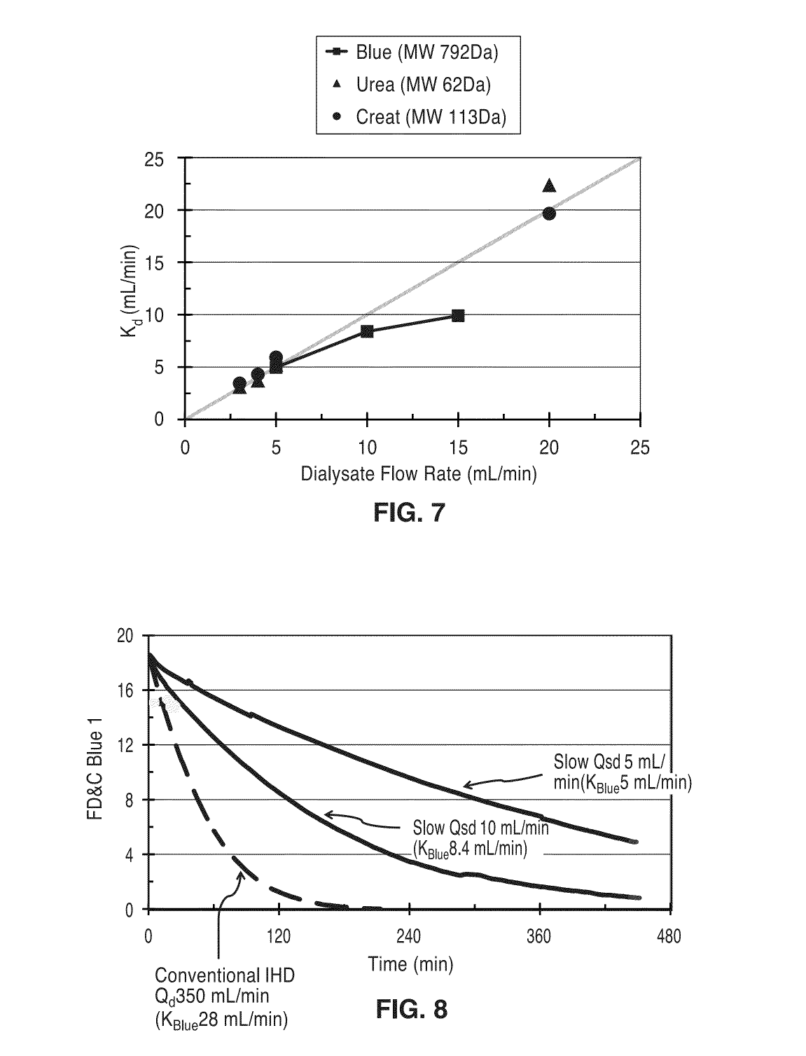

FIG. 7 is a graph showing the ex vitro dialyzer clearance (K_{d}) of FD&C Blue1 (792 Da, squares) and the in vivo dialyzer clearance of urea nitrogen (triangles) and creatinine (circles) in animal-patients dialyzed with the technology described herein. The clearances of both urea nitrogen and creatinine equaled the slow dialysate flow rate up to 20 mL/min. The clearance of FD&C Blue 1 decreased from linearity at flows greater than 5 mL/min, indicating the larger solute, FD&C Blue1, did not equilibrate completely with the dialysate at faster rate with the Fresenius Hemoflow™ F3 hemodialyzer.

FIG. 8 is a plot showing the real-time changes in FD&C Blue 1 during ex vivo hemodialysis experiments in “simulated patients.” Blood flow (Q_{b}) was 50 mL/min for all simulations. Dialysate flow (Q_{d}) was 350 mL/min in the conventional IHD simulations and reduced by the technology described herein to 5 and 10 mL/min for the slow-flow simulations. The concentration (C_{blue}) of FD&C Blue 1 was measured every 20 seconds for the 450 minute experiments. The intensity (rate) of dialysis of FD&C Blue 1 was substantially decreased during the slow-flow simulations compared to conventional IHD as shown by the slower rates of change in C_{Blue}, and decreases in the calculated clearance (K_{Blue}) at 5 and 10 mL/min. K_{blue} equaled Q_{d} at 5 mL/min but was slightly less than Q_{d} at 10 mL/min due to the large size (792 Da) of FD&C Blue1.

From the foregoing, one skilled in the art will readily appreciate that the systems and methods 10, 50, 100 and 150 of the technology described herein provide an inexpensive device that permits the ability to provide an equivalent slow continuous (dialytic) renal replacement therapy in addition to conventional IHD techniques on a single IHD dialysis platform, rather than requiring separate machines and platforms.

The systems and methods 10, 50, 100 and 150 of the technology described herein permit the very slow and precise delivery of hemodialysis at fast blood flow rates to minimize clotting in the extracorporeal circuit and the requirement for large doses of anticoagulant.

The systems and methods 10, 50, 100 and 150 also provide for delivery of dialysate-controlled hemodialysis with on-line generated standard or ultrapure dialysate, rather than with use of commercially prepared, sterile, and expensive dialysate solutions for greater treatment economy.

Still further, the systems and methods 10, 50, 100 and 150 require no intrinsic modification or reconfiguration of the conventional IHD machine and no loss of functionality, systems monitoring, or safety features of the conventional IHD machine. Additionally, the technology described herein permits direct prediction of the blood clearance for greater precision in the prescription and delivery of the hemodialysis treatment.

Embodiments of the present technology may be described with reference to flowchart illustrations of methods and systems according to embodiments of the technology, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that the terms “programming” or “program executable” as used herein refer to one or more instructions that can be executed by a processor to perform a function as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors. It will further be appreciated that as used herein, that the terms processor, computer processor, central processing unit (CPU), and computer are used synonymously to denote a device capable of executing the instructions and communicating with input/output interfaces and/or peripheral devices.

From the discussion above it will be appreciated that the technology described herein can be embodied in various ways, including the following:

1. An adaptor for providing slow dialysate flow with an intermittent hemodialysis (IHD) delivery system, the adaptor comprising: a dialysate slow-flow fluid pump; said pump having an outlet for connection to an inlet port of a hemodialyzer and an inlet for connection to an on-line generated dialysate bulk flow line in association with an IHD delivery system; and an outlet line for coupling an outlet port of the hemodialyzer back to the dialysate bulk flow line; wherein the dialysate slow-flow fluid pump is configured to control the rate of delivery of dialysate to the hemodialyzer.

2. The adaptor of any preceding embodiment: wherein the inlet port of the hemodialyzer comprises an inlet dialysate port at or near a blood outlet end of the hemodialyzer; and wherein the outlet port of the hemodialyzer comprises an outlet dialysate port at or near a blood inlet end of the hemodialyzer.

3. The adaptor of any preceding embodiment, further comprising: a sampling port located within the outlet line between the hemodialyzer and the dialysate bulk flow line.

4. The adaptor of any preceding embodiment, wherein the dialysate slow-flow fluid pump is configured to control the rate of delivery of dialysate to the hemodialyzer to a flow rate of less than 100 ml/min.

5. The adaptor of any preceding embodiment, wherein the dialysate slow-flow fluid pump is configured to control the rate of delivery of dialysate to the hemodialyzer to a slow-flow rate of less than 20 ml/min.

6. The adaptor of any preceding embodiment, wherein the dialysate slow-flow fluid pump is configured to control the rate of delivery of dialysate to the hemodialyzer to a slow-flow rate of less than 10 ml/min.

7. The adaptor of any preceding embodiment, further comprising: one or more of an inlet flow sensor or inlet conductivity sensor upstream of the dialysate slow-flow fluid pump.

8. The adaptor any preceding embodiment, further comprising: an inlet pressure sensor downstream of the dialysate slow-flow fluid pump.

9. The adaptor of any preceding embodiment, further comprising: one or more of an outlet pressure sensor, outlet dialysate flow sensor, and outlet conductivity sensor in the outlet line between the outlet port of the hemodialyzer and dialysate bulk flow line.

10. The adaptor of any preceding embodiment, wherein the dialysate slow-flow fluid pump comprises a variable speed pump.

11. A hemodialysis system for variably providing slow dialysate flow for continuous renal replacement therapy (CRRT) and high flow for intermittent hemodialysis (IHD) delivery, the system comprising: a hemodialyzer; a dialysate bulk flow line for use in association with an IHD delivery system; a dialysate slow-flow fluid pump; said pump having an outlet for connection to an inlet port of the hemodialyzer and an outlet for connection to a dialysate bulk flow line; and an outlet line for coupling an outlet port of the hemodialyzer back to the dialysate bulk flow line; wherein the dialysate slow-flow fluid pump is configured to control the rate of delivery of dialysate to the hemodialyzer.

12. The system of any preceding embodiment, wherein the inlet port of the hemodialyzer comprises an inlet dialysate port at or near a blood outlet end of the hemodialyzer; and wherein the outlet port of the hemodialyzer comprises an outlet dialysate port at or near a blood inlet end of the hemodialyzer.

13. The system of any preceding embodiment, further comprising: a sampling port located within the outlet line between the hemodialyzer and the dialysate bulk flow line.

14. The system of any preceding embodiment, wherein the dialysate slow-flow fluid pump is configured to control the rate of delivery of dialysate to the hemodialyzer to a flow rate of less than 100 ml/min.

15. The system of any preceding embodiment, wherein the dialysate slow-flow fluid pump is configured to control the rate of delivery of dialysate to the hemodialyzer to a slow-flow rate of less than 20 ml/min.

16. The system of any preceding embodiment, wherein the dialysate slow-flow fluid pump is configured to control the rate of delivery of dialysate to the hemodialyzer to a slow-flow rate of less than 10 ml/min.

17. The system of any preceding embodiment, further comprising: one or more of an inlet flow sensor or inlet conductivity sensor upstream of the dialysate slow-flow fluid pump.

18. The system of any preceding embodiment, further comprising: an inlet pressure sensor downstream of the dialysate slow-flow fluid pump.

19. The system of any preceding embodiment, further comprising: one or more of an outlet pressure sensor, outlet dialysate flow sensor, and outlet conductivity sensor in the outlet line between the outlet port of the hemodialyzer and dialysate bulk flow line.

20. The system of any preceding embodiment, wherein the dialysate slow-flow fluid pump comprises a variable speed pump.

21. A method for providing slow dialysate flow with an intermittent hemodialysis (IHD) delivery system, the method comprising: coupling an inlet of a dialysate slow-flow fluid pump to an on-line generated dialysate bulk flow line in association with an IHD delivery system; coupling an outlet of the dialysate slow-flow fluid pump to an inlet port of a hemodialyzer; coupling an outlet port of the hemodialyzer back to the dialysate bulk flow line; and controlling the rate of delivery of dialysate to the hemodialyzer.

22. The method of any preceding embodiment, wherein the inlet port of the hemodialyzer comprises an inlet dialysate port at or near a blood outlet end of the hemodialyzer; and wherein the outlet port of the hemodialyzer comprises an outlet dialysate port at or near a blood inlet end of the hemodialyzer.

23. The method of any preceding embodiment, further comprising: sampling the dialysate in an outlet line between the hemodialyzer and the dialysate bulk flow line.

24. The method of any preceding embodiment, wherein the dialysate slow-flow fluid pump is configured to control the rate of delivery of dialysate to the hemodialyzer to a flow rate of less than 100 ml/min.

25. The method of any preceding embodiment, wherein the dialysate slow-flow fluid pump is configured to control the rate of delivery of dialysate to the hemodialyzer to a slow-flow rate of less than 20 ml/min.

26. The method of any preceding embodiment, wherein the dialysate slow-flow fluid pump is configured to control the rate of delivery of dialysate to the hemodialyzer to a slow-flow rate of less than 10 ml/min.

27. The method of any preceding embodiment, further comprising: sensing one or more of the inlet flow, inlet conductivity, and inlet pressure of dialysate flow upstream form the hemodialyzer.

28. The method of any preceding embodiment, further comprising: sensing one or more of the outlet flow, outlet conductivity, and outlet pressure of dialysate flow downstream form the hemodialyzer.

29. The method of any preceding embodiment, wherein the dialysate comprises standard or ultra-pure dialysate.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An adaptor for providing slow dialysate flow with an intermittent hemodialysis (IHD) delivery system, the adaptor comprising:
a dialysate slow-flow fluid pump;
said pump having an outlet for connection to an inlet port of a hemodialyzer and an inlet for connection to an on-line generated dialysate bulk flow line in association with an IHD delivery system; and
an outlet line for coupling an outlet port of the hemodialyzer back to the dialysate bulk flow line;
wherein the dialysate slow-flow fluid pump is configured to control the rate of delivery of dialysate to the hemodialyzer.

2. An adaptor as recited in claim 1:
wherein the inlet port of the hemodialyzer comprises an inlet dialysate port at or near a blood outlet end of the hemodialyzer; and
wherein the outlet port of the hemodialyzer comprises an outlet dialysate port at or near a blood inlet end of the hemodialyzer.

3. An adaptor as recited in claim 1, further comprising:
a sampling port located within the outlet line between the hemodialyzer and the dialysate bulk flow line.

4. An adaptor as recited in claim 1, wherein the dialysate slow-flow fluid pump is configured to control the rate of delivery of dialysate to the hemodialyzer to a flow rate of less than 100 ml/min.

5. An adaptor as recited in claim 4, wherein the dialysate slow-flow fluid pump is configured to control the rate of delivery of dialysate to the hemodialyzer to a slow-flow rate of less than 20 ml/min.

6. An adaptor as recited in claim 5, wherein the dialysate slow-flow fluid pump is configured to control the rate of delivery of dialysate to the hemodialyzer to a slow-flow rate of less than 10 ml/min.

7. An adaptor as recited in claim 1, further comprising:
one or more of an inlet flow sensor or inlet conductivity sensor upstream of the dialysate slow-flow fluid pump.

8. An adaptor as recited in claim 7, further comprising:
an inlet pressure sensor downstream of the dialysate slow-flow fluid pump.

9. An adaptor as recited in claim 8, further comprising:
one or more of an outlet pressure sensor, outlet dialysate flow sensor, and outlet conductivity sensor in the outlet line between the outlet port of the hemodialyzer and dialysate bulk flow line.

10. An adaptor as recited in claim 4, wherein the dialysate slow-flow fluid pump comprises a variable speed pump.

11. A hemodialysis system for variably providing slow dialysate flow for continuous renal replacement therapy (CRRT) and high flow for intermittent hemodialysis (IHD) delivery, the system comprising:
a hemodialyzer;
a dialysate bulk flow line for use in association with an IHD delivery system;
a dialysate slow-flow fluid pump;
said pump having an outlet for connection to an inlet port of the hemodialyzer and an outlet for connection to a dialysate bulk flow line; and
an outlet line for coupling an outlet port of the hemodialyzer back to the dialysate bulk flow line;
wherein the dialysate slow-flow fluid pump is configured to control the rate of delivery of dialysate to the hemodialyzer.

12. A system as recited in claim 11:
wherein the inlet port of the hemodialyzer comprises an inlet dialysate port at or near a blood outlet end of the hemodialyzer; and
wherein the outlet port of the hemodialyzer comprises an outlet dialysate port at or near a blood inlet end of the hemodialyzer.

13. A system as recited in claim 11, further comprising:
a sampling port located within the outlet line between the hemodialyzer and the dialysate bulk flow line.

14. A system as recited in claim 11, wherein the dialysate slow-flow fluid pump is configured to control the rate of delivery of dialysate to the hemodialyzer to a flow rate of less than 100 ml/min.

15. A system as recited in claim 14, wherein the dialysate slow-flow fluid pump is configured to control the rate of delivery of dialysate to the hemodialyzer to a slow-flow rate of less than 20 ml/min.

16. A system as recited in claim 15, wherein the dialysate slow-flow fluid pump is configured to control the rate of delivery of dialysate to the hemodialyzer to a slow-flow rate of less than 10 ml/min.

17. A system as recited in claim 11, further comprising:
one or more of an inlet flow sensor or inlet conductivity sensor upstream of the dialysate slow-flow fluid pump.

18. A system as recited in claim 17, further comprising:
an inlet pressure sensor downstream of the dialysate slow-flow fluid pump.

19. A system as recited in claim 18, further comprising:
one or more of an outlet pressure sensor, outlet dialysate flow sensor, and outlet conductivity sensor in the outlet line between the outlet port of the hemodialyzer and dialysate bulk flow line.

20. A system as recited in claim 14, wherein the dialysate slow-flow fluid pump comprises a variable speed pump.

21. A method for providing slow dialysate flow with an intermittent hemodialysis (IHD) delivery system, the method comprising:
coupling an inlet of a dialysate slow-flow fluid pump to an on-line generated dialysate bulk flow line in association with an IHD delivery system;
coupling an outlet of the dialysate slow-flow fluid pump to an inlet port of a hemodialyzer;
coupling an outlet port of the hemodialyzer back to the dialysate bulk flow line; and
controlling the rate of delivery of dialysate to the hemodialyzer.

22. A method as recited in claim 21:
wherein the inlet port of the hemodialyzer comprises an inlet dialysate port at or near a blood outlet end of the hemodialyzer; and
wherein the outlet port of the hemodialyzer comprises an outlet dialysate port at or near a blood inlet end of the hemodialyzer.

23. A method as recited in claim 21, further comprising:
sampling the dialysate in an outlet line between the hemodialyzer and the dialysate bulk flow line.

24. A method as recited in claim 21, wherein the dialysate slow-flow fluid pump is configured to control the rate of delivery of dialysate to the hemodialyzer to a flow rate of less than 100 ml/min.

25. A method as recited in claim 24, wherein the dialysate slow-flow fluid pump is configured to control the rate of delivery of dialysate to the hemodialyzer to a slow-flow rate of less than 20 ml/min.

26. A method as recited in claim 25, wherein the dialysate slow-flow fluid pump is configured to control the rate of delivery of dialysate to the hemodialyzer to a slow-flow rate of less than 10 ml/min.

27. A method as recited in claim 1, further comprising:
sensing one or more of the inlet flow, inlet conductivity, and inlet pressure of dialysate flow upstream form the hemodialyzer.

28. A method as recited in claim 27, further comprising:
sensing one or more of the outlet flow, outlet conductivity, and outlet pressure of dialysate flow downstream form the hemodialyzer.

29. A method as recited in claim 21, wherein the dialysate comprises standard or ultra-pure dialysate.
